(19) United States
(12) Patent Application Publication
(10) Pub. No.: US 2020/0375867 A1
(43) Pub. Date: Dec. 3, 2020

(54) METHOD FOR SUPPRESSING DISCOLORATION OF EXTERNAL COMPOSITION CONTAINING ADENOSINE PHOSPHATE AND TRANEXAMIC ACID

(71) Applicant: OTSUKA PHARMACEUTICAL CO., LTD., Tokyo (JP)

(72) Inventors: Miyoko OGIHARA, Osaka-shi, Osaka (JP); Momoko SUMA, Osaka-shi, Osaka (JP); Shiori MIYAWAKI, Osaka-shi, Osaka (JP)

(73) Assignee: OTSUKA PHARMACEUTICAL CO., LTD., Tokyo (JP)

(21) Appl. No.: 16/966,259

(22) PCT Filed: Jan. 30, 2019
(86) PCT No.: PCT/JP2019/003058

§ 371 Date: Jul. 30, 2020

(30) Foreign Application Priority Data
Jan. 31, 2018 (JP) 2018-015486

(51) Int. Cl. A61K 8/60; A61K 8/44; A61Q 19/02; A61K 8/34; A61K 8/24; A61K 8/55; A61K 8/41; A61K 8/19

(52) U.S. Cl. CPC A61K 8/606; A61K 8/44; A61Q 19/02; A61K 8/19; A61K 8/24; A61K 8/55; A61K 8/41; A61K 8/345

(57) ABSTRACT

The present disclosure provides a composition for external use comprising an adenosine phosphate and/or a salt thereof and tranexamic acid and/or a salt thereof, which further comprises a particular chelating agent and/or a pH adjuster that is an organic alkali compound.

### TECHNICAL FIELD

The present application relates to a method for suppressing discoloration of a composition for external use comprising an adenosine phosphate and/or a salt thereof and tranexamic acid and/or a salt thereof, and to such composition suppressed in discoloration.

### BACKGROUND ART

An adenosine phosphate is known to have a moisturizing effect and a whitening effect, and cosmetics comprising an adenosine phosphate have already been put on the market.

Tranexamic acid has an anti-plasmin effect and is blended in cosmetics as an active ingredient for improving rough skin and whitening, and such cosmetics have already been on the market.

Patent Document 1 discloses a composition for preventing or improving pigmentation comprising an adenosine monophosphate and tranexamic acid.

### PRIOR ART DOCUMENT

### Patent Document

Patent Document 1: WO 2006/033343

The disclosures of the prior art documents cited herein are all incorporated herein by reference.

### SUMMARY OF INVENTION

### Technical Problem

The present inventors tried to develop a composition comprising an adenosine phosphate and tranexamic acid, and encountered the problem that the composition discolored with time (especially yellowing). An object of the present invention is to provide a method for suppressing discoloration with time in a composition comprising an adenosine phosphate and/or a salt thereof and tranexamic acid and/or a salt thereof, and to provide a composition in which the discoloration is suppressed.

### Solution to Problem

The present inventors have studied intensively to solve the above problem, and found that specific chelating agents and specific pH adjusters can respectively suppress the discoloration with time of a composition comprising an adenosine phosphate and/or a salt thereof and tranexamic acid and/or a salt thereof, thereby reaching the present invention. In addition, there has been a problem that such compositions provide a sticky feeling due to an adenosine phosphate and/or a salt thereof, and the present inventors have found that it is preferable to blend specific ingredient(s) (ingredients for suppressing sticky feeling) in order to suppress the sticky feeling, and further found that some specific ingredient(s) among these ingredients for suppressing sticky feeling do not deteriorate the discoloration with time.

Thus, the present application provides the following:

[1] A composition for external use, comprising:

Ingredient (A): an adenosine phosphate and/or a salt thereof; and

Ingredient (B): tranexamic acid and/or a salt thereof, and further comprising

Ingredient (C): a chelating agent selected from edetic acid or a salt thereof, a phosphate compound, and a mixture thereof; and/or

Ingredient (D): a pH adjuster which is an organic alkali compound.

[2] The composition according to [1], wherein the adenosine phosphate and/or a salt thereof is an adenosine monophosphate and/or a salt thereof.
[3] The composition according to [1] or [2], wherein the phosphate compound is selected from tripolyphosphoric acid or a salt thereof, metaphosphoric acid or a salt thereof, etidronic acid or a salt thereof, phytic acid or a salt thereof, and a mixture thereof.
[4] The composition according to any one of [1] to [3], wherein Ingredient (C) is the phosphate compound selected from tripolyphosphoric acid or a salt thereof, metaphosphoric acid or a salt thereof, etidronic acid or a salt thereof, and a mixture thereof.
[5] The composition according to any one of [1] to [4], wherein Ingredient (D) is selected from aminohydroxymethylpropanediol, aminomethylpropanol, aminomethylpropanediol, arginine, triethanolamine, and a mixture thereof.
[6] The composition according to any one of [1] to [5], wherein Ingredient (C) is comprised in a weight ratio of (Ingredient (A)+Ingredient (B)):Ingredient (C)=1:0.0001 to 0.5.
[7] The composition according to any one of [1] to [6], which comprises Ingredient (D) and has pH of 5.5 to 7.5.
[8] The composition according to any one of [1] to [7], wherein Ingredient (D) is comprised in a weight ratio of Ingredient (A):Ingredient (D)=1:0.00001 to 4.
[9] The composition according to any one of [1] to [8], which comprises 0.01 to 10% by weight of Ingredient (A).
[10] The composition according to any one of [1] to [9], which comprises 0.01 to 10% by weight of Ingredient (B).
[11] The composition according to any one of [1] to [10], further comprising Ingredient (E): bis-ethoxydiglycol cyclohexane dicarboxylate, diethoxyethyl succinate, highly polymerized polyethylene glycol, highly polymerized silicone, pullulan, polyvinylpyrrolidone, an organic fine particle, biosaccharide gum-1, or a mixture thereof.
[12] A method for producing a composition for external use, comprising mixing Ingredient (A): an adenosine phosphate and/or a salt thereof; and Ingredient (B): tranexamic acid and/or a salt thereof, in the presence of Ingredient (C): a chelating agent selected from edetic acid or a salt thereof, a phosphate compound, and a mixture thereof; and/or Ingredient (D): a pH adjuster which is an organic alkali compound.
[13] A method for suppressing discoloration of a composition for external use which comprises Ingredient (A): an adenosine phosphate and/or a salt thereof; and Ingredient (B): tranexamic acid and/or a salt thereof,

comprising using Ingredient (C): a chelating agent selected from edetic acid or a salt thereof, a phosphate compound, and a mixture thereof; and/or Ingredient (D): a pH adjuster which is an organic alkali compound.

### Effect of the Invention

The present invention provides a composition for external use, comprising an adenosine phosphate and/or a salt thereof, and tranexamic acid and/or a salt thereof, in which the discoloration with time is suppressed.

### DESCRIPTION OF EMBODIMENT

The present application relates to a composition for external use, comprising

Ingredient (A): an adenosine phosphate and/or a salt thereof; and Ingredient (B): tranexamic acid and/or a salt thereof; and further comprising

Ingredient (C): a chelating agent selected from edetic acid or a salt thereof, a phosphate compound, and a mixture thereof; and/or

Ingredient (D): a pH adjuster which is an organic alkali compound.

The composition comprising an adenosine phosphate and/or a salt thereof and tranexamic acid and/or a salt thereof may be inhibited from discoloration with time by compounding Ingredient (C) and/or Ingredient (D).

In the present application, examples of an adenosine phosphate include adenosine monophosphate (adenosine 2′-monophosphate, adenosine 3′-monophosphate, adenosine 5′-monophosphate, etc.), adenosine diphosphate (adenosine 5′-diphosphate, etc.), adenosine triphosphate (adenosine 5′-triphosphate, etc.), adenosine 3′,5′-cyclic phosphate, and the like, and a single kind of the adenosine phosphate may be used, or any combination of two or more kinds of the adenosine phosphate may be used.

In the present application, a salt of adenosine phosphate(s) is not particularly limited as long as it can be formulated in a cosmetic, a drug or quasi-drug for external use. Examples of a salt of adenosine monophosphate include specifically alkali metal salts such as sodium salts, potassium salts and the like; alkaline earth metal salts such as calcium salts, magnesium salts, barium salts and the like; basic amino acid salts such as arginine, lysine and the like; ammonium salts such as ammonium salts, tricyclohexylammonium salts and the like; alkanolamine salts such as monoethanolamine salts, diethanolamine salts, triethanolamine salts, monoisopropanolamine salts, diisopropanolamine salts, triisopropanolamine and the like, and a single kind of the salt of adenosine phosphate(s) may be used, or any combination of two or more kinds of the salt of adenosine phosphate(s) may be used.

The amount of Ingredient (A) included in the composition of the present application may vary depending on the use, form, etc. of the composition for external use, but may be optionally selected from a range of, for example, usually 0.001 to 10% by weight based on the total weight of the composition for external use. Preferably from 0.01% by weight to 10% by weight, more preferably from 0.1% by weight to 7% by weight, more preferably from 0.5% by weight to 5% by weight, still more preferably from 0.7% by weight to 3% by weight, and even more preferably from 1% by weight to 2% by weight are exemplified.

Further examples of the lower limit of the amount range of Ingredient (A) included in the composition for external use of the application include 0.001% by weight, 0.01% by weight, 0.1% by weight, 0.5% by weight, 0.7% by weight, and 1% by weight, and examples of the upper limit include 1% by weight, 2% by weight, 3% by weight, 5% by weight, 7% by weight, and 10% by weight, and preferred examples of the range may be shown by a combination of the lower limit and the upper limit.

In the present application, a salt of tranexamic acid is not particularly limited as long as it can be formulated in a cosmetic, a drug or quasi-drug for external use. Examples of a salt of tranexamic acid include alkali metal salts, alkaline earth metal salts, basic amino acid salts, ammonium salts, alkanolamine salts, and the like, as mentioned above for an adenosine phosphate, and a single kind of salt of tranexamic acid may be used, or any combination of two or more kinds of salt of tranexamic acid may be used.

The amount of Ingredient (B) included in the composition of the present application may vary depending on the use, form, etc. of the composition for external use, but may be optionally selected from, for example, a range of usually 0.001 to 10% by weight based on the total weight of the composition for external use. Preferably from 0.01% by weight to 10% by weight, more preferably from 0.1% by weight to 7% by weight, more preferably from 0.5% by weight to 5% by weight, still more preferably from 0.7% by weight to 3% by weight, and even more preferably from 1% by weight to 2% by weight are exemplified.

Further, examples of the lower limit of the amount range of Ingredient (B) included in the composition for external use of the application include 0.001% by weight, 0.01% by weight, 0.1% by weight, 0.5% by weight, 0.7% by weight, and 1% by weight, and examples of the upper limit include 1% by weight, 2% by weight, 3% by weight, 5% by weight, 7% by weight, and 10% by weight, and preferred examples of the range may be indicated by a combination of the lower limit and the upper limit.

Examples of the chelating agent for Ingredient (C) includes edetic acid or a salt thereof and a phosphate compound (for example, tripolyphosphoric acid or a salt thereof, metaphosphoric acid or a salt thereof, etidronic acid or a salt thereof, and phytic acid or a salt thereof), and a single kind of the chelating agent may be used, or any combination of two or more kinds of the chelating agent may be used. Preferred examples of the chelating agent of Ingredient (C) include tripolyphosphoric acid or a salt thereof, metaphosphoric acid or a salt thereof, and etidronic acid or a salt thereof. In the composition of the present application, chelating agent(s) other than Ingredient (C) may be used to the extent that discoloration with time is not problematic.

Examples of a salt of edetic acid include EDTA-2K, EDTA-2Na, EDTA-3K, EDTA-3Na, EDTA-4Na, EDTA (Ca/2Na) and the like. A single kind of salt of edetic acid may be used, or any combination of two or more kinds of salt of edetic acid may be used. Preferred examples of edetic acid or a salt thereof include EDTA, EDTA-2Na, EDTA-3Na and EDTA-4Na.

Examples of a salt of tripolyphosphoric acid, a salt of metaphosphoric acid, a salt of etidronic acid, and a salt of phytic acid include alkali metal salts, alkaline earth metal salts, basic amino acid salts, ammonium salts, alkanolamine salts, and the like, as mentioned above for an adenosine phosphate. A single kind of the salt of these may be used, or any combination of two or more kinds of the salt of these may be used.

Preferred examples of a salt of tripolyphosphoric acid include sodium tripolyphosphate and potassium tripolyphosphate.

Preferred examples of a salt of metaphosphoric acid include sodium metaphosphate and potassium metaphosphate.

Preferred examples of a salt of etidronic acid include trisodium etidronate, tetrapotassium etidronate, and tetrasodium etidronate.

Preferred examples of a salt of phytic acid include calcium phytate and sodium phytate.

In the composition for external use of the present application, the amount of Ingredient (C) is not particularly limited as long as the discoloration with time of the composition is suppressed, but it is preferable that Ingredient (C) is comprised in a weight ratio of 0.00001 to 0.5 (more preferably 0.0001 to 0.2, still more preferably 0.0002 to 0.1) based on the sum of Ingredient (A) and Ingredient (B), that is, (Ingredient (A)+Ingredient (B)):Ingredient (C)=1:0.00001 to 0.5 (more preferably 1:0.0001 to 0.2, still more preferably 1:0.0002 to 0.1). Further, examples of the lower limit value of the weight ratio range of Ingredient (C) when the total weight of Ingredient (A) and Ingredient (B) comprised in the composition for external use of the present application is 1 include 0.00001, 0.0001, 0.0002, 0.0005, 0.001, 0.01, 0.05, examples of the upper limit value include 0.05, 0.1, 0.2, 0.5, and preferred examples of the range may be indicated by a combination of the lower limit value and the upper limit value.

The amount of Ingredient (C) that may be comprised in the composition of the present application may be optionally selected, for example, from a range of usually 0.0001 to 2% by weight based on the total weight of the composition for external use. Preferably 0.0002 to 1.5% by weight, more preferably 0.0005 to 1% by weight, and more preferably 0.001 to 0.5% by weight are exemplified.

Further examples of the lower limit of the amount range of Ingredient (C) that may be included in the composition for external use of the application include 0.0001% by weight, 0.0002% by weight, 0.0005% by weight, 0.001% by weight, 0.01% by weight, 0.1% by weight, and 0.2% by weight, and examples of the upper limit include 0.2% by weight, 0.5% by weight, 1% by weight, and 2% by weight, and preferred examples of the range may be indicated by a combination of the lower limit and the upper limit.

Examples of a pH adjuster which is an organic alkali compound of Ingredient (D) include aminohydroxymethylpropanediol, aminomethylpropanol, aminomethylpropanediol, arginine, and triethanolamine, and a single kind of the pH adjuster may be used, or any combination of two or more kinds of the pH adjuster may be used. Preferred examples of a pH adjuster which is an organic alkali compound include aminohydroxymethylpropanediol, aminomethylpropanediol, and arginine.

In order to suppress discoloration with time, it is preferable to use the pH adjuster of Ingredient (D) for adjusting the pH of the composition for external use of the present application. The preferred pH of the composition for external use of the present application may vary depending on the use of the composition and the like, but may be pH 5.5 to 7.5. In order to adjust pH of the composition for external use of the present application, a pH adjuster other than Ingredient (D) may be used to the extent that discoloration with time is not problematic.

In the composition for external use of the present application, the amount of Ingredient (D) is not particularly limited as long as the discoloration with time of the composition is suppressed, and may vary depending on the amount of Ingredient (A), the kind and amount of optional ingredient(s), and the preferable pH of the composition, but for example, Ingredient (D) may be comprised in a weight ratio of 0.0001 to 4 (more preferably 0.001 to 2, still more preferably 0.005 to 1) based on the Ingredient (A), that is, Ingredient (A):Ingredient (D)=1:0.0001 to 4 (more preferably 1:0.001 to 2, still more preferably 1:0.005 to 1).

Further, examples of the lower limit of the weight ratio range of Ingredient (C) when the weight of Ingredient (A) comprised in the composition for external use of the present application is 1 include 0.0001, 0.001, 0.01, and 0.1, examples of the upper limit include 0.1, 0.5, 1, 2, 3, and 4, and preferred examples of the range may be indicated by a combination of the lower limit and the upper limit.

The composition suppressed in sticky feeling and suppressed in discoloration with time can be provided by compounding Ingredient (E): bis-ethoxydiglycol cyclohexane dicarboxylate, diethoxyethyl succinate, highly polymerized polyethylene glycol (for example, those with a freezing point of 40° C. or higher), highly polymerized silicone (for example, those with a kinematic viscosity of 6000 mm²/s or more), pullulan, polyvinylpyrrolidone, an organic fine particle (for example, silica, poly(methyl methacrylate), talc, and urethane), biosaccharide gum-1 or a mixture thereof.

In order to provide better suppression of sticky feeling in the composition for external use of the present application, it is preferable that Ingredient (A) and Ingredient (E) are comprised in a weight ratio of Ingredient (A):Ingredient (E)=1:0.01 to 30 (more preferably 1:0.02 to 25, still more preferably 1:0.1 to 20).

Further, examples of the lower limit of the range of the weight ratio of Ingredient (E) when the weight of Ingredient (A) comprised in the composition for external use of the present application is 1 include 0.01, 0.03, 0.05, 0.1, 0.5, 0.7, 1, examples of the upper limit include 1.5, 2, 3, 5, 10, 20, 25, 30, and preferred examples of the range may be indicated by a combination of the lower limit and the upper limit.

The amount of Ingredient (E) included in the composition of the present application may vary depending on the kind of Ingredient (E) to be used and the use, form, etc. of the composition for external use, but may be optionally selected from a range of, for example, usually 0.001 to 10% by weight based on the total weight of the composition for external use. Preferably from 0.01% by weight to 10% by weight, more preferably from 0.1% by weight to 7% by weight, more preferably from 0.5% by weight to 5% by weight, and even more preferably from 0.7% by weight to 2% by weight are exemplified.

Further, examples of the lower limit of the amount range of Ingredient (E) included in the composition for external use of the present application include 0.001% by weight, 0.01% by weight, 0.1% by weight, 0.5% by weight, 0.7% by weight, and 1% by weight, and examples of the upper limit include 1% by weight, 2% by weight, 3% by weight, 5% by weight, 7% by weight, and 10% by weight, and preferred examples of the range may be indicated by a combination of the lower limit and the upper limit.

Each ingredient that may be compounded into the composition of the present application may be in the form of a hydrate.

The composition for external use of the present application may be prepared in various forms by combining pharmaceutically or cosmetically acceptable base(s) or carrier(s) in addition to the above ingredients. For pharmaceutically or cosmetically acceptable base(s) and carrier(s), conventionally known one(s) can be used. The composition of the present invention may comprise, if required, a wide variety of known ingredients used for externally-applied compositions suitable for the skin or mucous membranes, such as cosmetics, externally-applied medical/quasi-medical drugs, etc. Examples of such ingredients include surfactants, colorants (dyes and pigments), flavors, preservatives, bactericides (antibacterials), thickeners, antioxidants, sequestering agents, cooling agents, deodorizers, humectants, UV absorbers, UV dispersants, vitamins, plant extracts, skin astringents, anti-inflammatory agents (antiphlogistic agents), whitening agents, cell activators, vasodilators, blood circulation accelerators, skin function accelerators, and the like.

For example, the composition for external use of the present application may optionally further comprise water, ethanol, glycerin, BG, 1,2-pentanediol, menthol, POE hydrogenated castor oil, phenoxyethanol, flavors, and a mixture thereof.

The composition for external use of the present invention may be used as a composition for external use to be applied or sprayed on the skin. Specifically, the composition of the present invention may be used as an external preparation (skin preparation) for cosmetics, external medicines or external quasi-medicines.

The form of the composition for external use of the present invention is not particularly limited as long as it can be applied to the skin, and examples thereof include a paste, a mousse, a gel, a liquid, a milky liquid, a suspension liquid, a cream, an ointment, a solid, a sheet, an aerosol, a spray, and a liniment. Especially when using as a cosmetic, lotion; emulsions such as emollient emulsion, milky lotion, nourishing emulsion, and cleansing emulsion; cream such as emollient cream, massage cream, cleansing cream, and makeup cream; a lip balm and the like.

### EXAMPLE

The present invention is explained in further detail with reference to Formulation Examples and Test Examples. However, the scope of the invention is not limited to these Examples.

### <Test Example 1> Chelating Agent for Suppressing Discoloration—I

The ingredients as shown in the following table were mixed and adjusted to pH 5.5 to 7.5 with potassium hydroxide to provide the comparative example and each of the formulation examples, which were filled in a closed glass container and stored at 60° C. for 2 weeks.

After the storage, a visual check was conducted to evaluate discoloration as below,

⊚: Not discolored;
∘: Almost not discolored;
Δ: Slightly discolored;
X: Markedly discolored.

The results are shown in the table below.

TABLE 1

| | Comparative | Formulation | Formulation | Formulation | Formulation | Formulation | Formulation |
|---|---|---|---|---|---|---|---|
| | example | example 1 | example 2 | example 3 | example 4 | example 5 | example 6 |
| Water | Balance | Balance | Balance | Balance | Balance | Balance | Balance |
| Adenosine | 2 | 2 | 2 | 2 | 2 | 2 | 2 |
| monophosphate | | | | | | | |
| Tranexamic acid | 2 | 2 | 2 | 2 | 2 | 2 | 2 |
| 1,2-Pentanediol | 1 | 1 | 1 | 1 | 1 | 1 | 1 |
| Sodium | | 0.001 | 0.005 | 0.01 | 0.05 | 0.1 | 0.2 |
| tripolyphosphate | | | | | | | |
| Result | X | ◯ | ⊚ | ⊚ | ⊚ | ⊚ | ⊚ |

| | Formulation | Formulation | Formulation | Formulation | Formulation | Formulation | Formulation | Formulation |
|---|---|---|---|---|---|---|---|---|
| | example 7 | example 8 | ex ample 9 | example 10 | example 11 | example 12 | example 13 | example 14 |
| Water | Balance | Balance | Balance | Balance | Balance | Balance | Balance | Balance |
| Adenosine | 2 | 2 | 2 | 2 | 2 | 2 | 2 | 2 |
| monophosphate | | | | | | | | |
| Tranexamic acid | 2 | 2 | 2 | 2 | 2 | 2 | 2 | 2 |
| 1,2-Pentanediol | 1 | 1 | 1 | 1 | 1 | 1 | 1 | 1 |
| Sodium | 0.01 | 0.05 | 0.1 | 0.2 | | | | |
| metaphosphate | | | | | | | | |
| Etidronic acid | | | | | 0.06 | | | |
| Phytic acid | | | | | | 0.1 | | |
| EDTA•2Na•2H₂O | | | | | | | 0.2 | |
| EDTA•4Na•4H₂O | | | | | | | | 0.2 |
| Result | ⊚ | ⊚ | ⊚ | ⊚ | ⊚ | ◯ | ◯ | ◯ |

### <Test Example 2> Chelating Agent for Suppressing Discoloration—II

The ingredients as shown in the following table were mixed and adjusted to pH 5.5 to 7.5 with potassium hydroxide to prepare the comparative example and each of the formulation examples, which were filled in a closed glass container and stored at 50° C. for 6 months.

After the storage, a visual check was conducted to evaluate discoloration as below,

⊚: Not discolored;
∘: Almost not discolored;
Δ: Slightly discolored;
X: Markedly discolored.

The results are shown in the table below.

“Other ingredients” in the comparative example and each of the formulation examples consists of the same ingredients: humectants (glycerin, butylene glycol, and 1,2-pentanediol), touch improver (bis-ethoxydiglycol cyclohexane dicarboxylate), preservative (phenoxyethanol), solubilizer (POE hardened castor oil), and flavor, and the comparative example and each of the formulation examples comprised the same amount of “Other ingredients”.

TABLE 2

| | Comparative | Formulation | Formulation | Formulation | Formulation | Formulation |
|---|---|---|---|---|---|---|
| | example | example 15 | example 16 | example 17 | example 18 | example 19 |
| Water | Balance | Balance | Balance | Balance | Balance | Balance |
| Adenosine | 1 | 1 | 1 | 1 | 1 | 1 |
| monophosphate | | | | | | |
| Tranexamic acid | 2 | 2 | 2 | 2 | 2 | 2 |
| Ethanol | 7 | 7 | 7 | 7 | 7 | 7 |
| Other ingredients | Suitable | Suitable | Suitable | Suitable | Suitable | Suitable |
| | amount | amount | amount | amount | amount | amount |
| Sodium | | 0.2 | | | | |
| tripolyphosphate | | | | | | |
| Sodium | | | 0.2 | | | |
| metaphosphate | | | | | | |
| Etidronic acid | | | | 0.2 | | |
| Phytic acid | | | | | 0.2 | |
| EDTA•2Na•2H₂O | | | | | | 0.2 |
| Result | X | ⊚ | ⊚ | ⊚ | ◯ | ◯ |

### <Test Example 3> pH Adjuster for Suppressing Discoloration

The ingredients as shown in the following table were mixed to provide the comparative example and each of the formulation examples (pH of these examples were 5.5 to 7.5), which were filled in a closed glass container and stored at 60° C. for 2 weeks.

After the storage, a visual check was conducted to evaluate discoloration as below,

⊚: Not discolored;
∘: Almost not discolored;
Δ: Slightly discolored;
X: Markedly discolored.

The results are shown in the table below.

TABLE 3

| | Formulation | Formulation | Formulation | Formulation | Comparative | |
|---|---|---|---|---|---|---|
| | example 20 | example 21 | example 22 | example 23 | example | |
| Water | Balance | Balance | Balance | Balance | Balance | Balance |
| Adenosine | 2 | 2 | 2 | 2 | 2 | 2 |
| monophosphate | | | | | | |
| Tranexamic acid | 2 | 2 | 2 | 2 | 2 | 2 |
| Phenoxyethanol | 0.2 | 0.2 | 0.2 | 0.2 | 0.2 | 0.2 |
| Aminohydroxymethylpropanediol | 1.1 | | | | | |
| Aminomethylpropanediol | | 1.1 | | | | |
| Arginine | | | 1.9 | | | |
| Triethanolamine | | | | 1.6 | | |
| Potassium hydroxide | | | | | 0.6 | |
| Sodium hydroxide | | | | | | 0.3 |
| Result | ⊚ | ⊚ | ⊚ | ◯ | X | X |

### <Example 4> Agent for Suppressing Sticky Feeling and not Accelerating Discoloration

The ingredients as shown in the following table were mixed and adjusted to pH 5.5 to 7.5 with potassium hydroxide to prepare the comparative example and each of the formulation examples. In “Other ingredients”, a preservative (phenoxyethanol), a solubilizing agent and a flavor were comprised.

### (Evaluation of Sticky Feeling)

The comparative example and each of the formulation examples (0.5 g) were applied to the faces of three panelists. The sticky feelings of the formulation examples were evaluated as below.

⊚: Suppressed compared with the comparative example;
∘: Slightly suppressed compared with the comparative example;
x: Not suppressed compared with the comparative example;

The results are shown in Table 4.

### (Evaluation of Discoloration)

The comparative example and each of the formulation examples were filled in a closed glass container and stored at 50° C. for 1 week or 40° C. for 6 months.

After the storage, a visual check was conducted to evaluate discoloration as below,

⊚: Not discolored;
∘: Almost not discolored;
Δ: Slightly discolored;
X: Markedly discolored.

The results are shown in Table 4.

TABLE 4

| | Comparative | Formulation | Formulation | Formulation | Formulation |
|---|---|---|---|---|---|
| % by weight | example | example 24 | example 25 | example 26 | example 27 |
| Water | Balance | Balance | Balance | Balance | Balance |
| Adenosine | | | | | |
| monophosphate | 2 | 2 | 2 | 2 | 1 |
| Tranexamic acid | 2 | 2 | 2 | 2 | 2 |
| Ethanol | 7 | 7 | 7 | 7 | 7 |
| Sodium | | | | | |
| tripolyphosphate | 0.2 | 0.2 | 0.2 | 0.2 | 0.2 |
| Other | Suitable | Suitable | Suitable | Suitable | Suitable |
| ingredients | amount | amount | amount | amount | amount |
| Bis-ethoxydiglycol | | 0.5 | | | |
| cyclohexane | | | | | |
| dicarboxylate | | | | | |
| Diethoxyethyl succinate | | | 1 | | |
| Highly polymerized | | | | 2 | |
| polyethylene glycol | | | | | |
| Highly polymerized | | | | | 0.02 |
| dimethicone | | | | | |
| Pullulan | | | | | |
| Polyvinylpyrrolidone | | | | | |
| Silica | | | | | |
| poly(methyl | | | | | |
| methacrylate) | | | | | |
| Visual check | ⊚ | ⊚ | ⊚ | ⊚ | ⊚ |
| (50° C. for 1 | | | | | |
| week) | | | | | |
| Visual check | ◯ | ◯ | ◯ | ◯ | ◯ |
| (40° C. for 6 | | | | | |
| months) | | | | | |
| Sticky feeling | X | ⊚ | ⊚ | ⊚ | ⊚ |

| | Formulation | Formulation | Formulation | Formulation |
|---|---|---|---|---|
| % by weight | example 28 | example 29 | example 30 | example 31 |
| Water | Balance | Balance | Balance | Balance |
| Adenosine | | | | |
| monophosphate | 1 | 1 | 1 | 1 |
| Tranexamic acid | 2 | 2 | 2 | 2 |
| Ethanol | 7 | 7 | 7 | 7 |
| Sodium | | | | |
| tripolyphosphate | 0.2 | 0.2 | 0.2 | 0.2 |
| Other | Suitable | Suitable | Suitable | Suitable |
| ingredients | amount | amount | amount | amount |
| Bis-ethoxydiglycol | | | | |
| cyclohexane | | | | |
| dicarboxylate | | | | |
| Diethoxyethyl succinate | | | | |
| Highly polymerized | | | | |
| polyethylene glycol | | | | |
| Highly polymerized | | | | |
| dimethicone | | | | |
| Pullulan | 0.1 | | | |
| Polyvinylpyrrolidone | | 0.1 | | |
| Silica | | | 2 | |
| poly(methyl | | | | 2 |
| methacrylate) | | | | |
| Visual check | ⊚ | ⊚ | ⊚ | ⊚ |
| (50° C. for 1 | | | | |
| week) | | | | |
| Visual check | ◯ | ◯ | ◯ | ◯ |
| (40° C. for 6 | | | | |
| months) | | | | |
| Sticky feeling | ⊚ | ⊚ | ⊚ | ⊚ |

| | Formulation | Formulation | Formulation | Formulation | Formulation |
|---|---|---|---|---|---|
| % by weight | example 32 | example 33 | example 34 | example 35 | example 36 |
| Water | Balance | Balance | Balance | Balance | Balance |
| Adenosine monophosphate | 1 | 1 | 2 | 2 | 2 |
| Tranexamic acid | 2 | 2 | 2 | 2 | 2 |
| Ethanol | 7 | 7 | 7 | 7 | 7 |
| Sodium tripolyphosphate | 0.2 | 0.2 | 0.2 | 0.2 | 0.2 |
| Other | Suitable | Suitable | Suitable | Suitable | Suitable |
| ingredients | amount | amount | amount | amount | amount |
| Talc | 2 | | | | |
| Urethane | | 2 | | | |
| Biosaccharide gum-1 | | | 0.05 | | |
| PEG/PPG/Polybutylene | | | | 1 | |
| glycol 8/5/3 | | | | | |
| glycerin | | | | | |
| Diglycerin | | | | | 3 |
| Polyglycerin | | | | | |
| Polyoxypropylene | | | | | |
| diglyceryl ether | | | | | |
| Polyoxyethylene methyl | | | | | |
| glucoside | | | | | |
| Polyglyceryl-10 | | | | | |
| Eicosanedioate/ | | | | | |
| Tetradecanedioate | | | | | |
| Discoloration | ⊚ | ⊚ | ⊚ | ◯ | Δ |
| (50° C. for 1 | | | | | |
| week) | | | | | |
| Discoloration | ◯ | ◯ | ◯ | Δ | — |
| (40° C. for 6 | | | | | |
| months) | | | | | |
| Sticky feeling | ⊚ | ⊚ | ⊚ | ⊚ | ⊚ |

| | Formulation | Formulation | Formulation | Formulation |
|---|---|---|---|---|
| % by weight | example 37 | example 38 | example 39 | example 40 |
| Water | Balance | Balance | Balance | Balance |
| Adenosine | 1 | 2 | 2 | 1 |
| monophosphate | | | | |
| Tranexamic | 2 | 2 | 2 | 2 |
| acid | | | | |
| Ethanol | 7 | 7 | 7 | 7 |
| Sodium | 0.2 | 0.2 | 0.2 | 0.2 |
| tripolyphosphate | | | | |
| Other | Suitable | Suitable | Suitable | Suitable |
| ingredients | amount | amount | amount | amount |
| Talc | | | | |
| Urethane | | | | |
| Biosaccharide gum-1 | | | | |
| PEG/PPG/Polybutylene | | | | |
| glycol 8/5/3 | | | | |
| glycerin | | | | |
| Diglycerin | | | | |
| Polyglycerin | 1 | | | |
| Polyoxypropylene | | 1 | | |
| diglyceryl ether | | | | |
| Polyoxyethylene methyl | | | 1 | |
| glucoside | | | | |
| Polyglyceryl-10 | | | | 1.5 |
| Eicosanedioate/ | | | | |
| Tetradecanedioate | | | | |
| Discoloration | Δ | Δ | Δ | Δ |
| (50° C. for 1 | | | | |
| week) | | | | |
| Discoloration | — | — | — | — |
| (40° C. for 6 | | | | |
| months) | | | | |
| Sticky feeling | ⊚ | ⊚ | ⊚ | ⊚ |

Formulation examples comprising 1% by weight, 1.5% by weight or 2% by weight of bis-ethoxydiglycol cyclohexane dicarboxylate were also tested. The results were the same as the formulation example comprising 0.5% by weight of bis-ethoxydiglycol cyclohexane dicarboxylate shown in Table 4.

Formulation examples comprising 1.5% by weight or 2% by weight of diethoxyethyl succinate were also tested. The results were the same as the formulation example comprising 1% by weight of diethoxyethyl succinate shown in Table 4.

Formulation examples comprising 2.5% by weight, 3% by weight, 3.5% by weight, 4% by weight, 4.5% by weight or 5% by weight of silica were also tested. The results were the same as the formulation example comprising 2% by weight of silica shown in Table 4.

Formulation examples comprising 2.5% by weight, 3% by weight, 3.5% by weight, 4% by weight, 4.5% by weight or 5% by weight of poly(methyl methacrylate) were also tested. The results were the same as the formulation example comprising 2% by weight of poly(methyl methacrylate) shown in Table 4.

Formulation examples comprising 2.5% by weight, 3% by weight, 3.5% by weight, 4% by weight, 4.5% by weight or 5% by weight of talc were also tested. The results were the same as the formulation example comprising 2% by weight of talc shown in Table 4.

Formulation examples comprising 2.5% by weight, 3% by weight, 3.5% by weight, 4% by weight, 4.5% by weight or 5% by weight of urethane were also tested. The results were the same as the formulation example comprising 2% by weight of urethane shown in Table 4.

Formulation examples comprising 1.5% by weight, 2% by weight, 2.5% by weight or 3% by weight of PEG/PPG/Polybutylene glycol-8/5/3 glycerin were also tested. The results were the same as the formulation example comprising 1% by weight of PEG/PPG/Polybutylene glycol-8/5/3 glycerin shown in Table 4.

Formulation examples comprising 1.5% by weight, 2% by weight, 2.5% by weight, 3% by weight, 3.5% by weight, 4% by weight, 4.5% by weight, or 5% by weight of polyglycerin were also tested. The results were the same as the formulation example comprising 1% by weight of polyglycerin shown in Table 4.

Formulation examples comprising 1.5% by weight, 2% by weight, 2.5% by weight, 3% by weight, 3.5% by weight, 4% by weight, 4.5% by weight, or 5% by weight of polyoxypropylene diglyceryl ether were also tested. The results were the same as the formulation example comprising 1% by weight of polyoxypropylene diglyceryl ether shown in Table 4.

Formulation examples comprising 2% by weight, 2.5% by weight, 3% by weight, 3.5% by weight, 4% by weight, 4.5% by weight, or 5% by weight of Polyglyceryl-10 Eicosanedioate/Tetradecanedioate were also tested. The results were the same as the formulation example comprising 1.5% by weight of Polyglyceryl-10 Eicosanedioate/Tetradecanedioate shown in Table 4.

### Formulation Example 41: Skin Lotion

### <Ingredients> % by Weight

| | |
|---|---|
| Adenosine phosphate | 1.5 |
| Tranexamic acid | 1.5 |
| Glycerin | 8 |
| Butylene glycol | 4 |
| Hydroxylated lecithin | 0.1 |
| Hyaluronic acid | 0.02 |
| Polyoxyethylene polyoxypropylene | 0.2 |
| decyl tetradecyl ether | |
| Etidronic acid | 0.02 |
| Aminomethylpropanediol | Suitable amount |
| Preservative | Suitable amount |
| Flavor | Suitable amount |
| Purified water | Balance |

### <Manufacturing Method>

According to a conventional method, the water-soluble ingredients were dissolved in purified water to provide a main phase. The water-insoluble ingredient mixed with the surfactants were added to the main phase to provide the skin lotion.

### Formulation Example 42: White-Turbid Skin Lotion

### <Ingredients> % by Weight

| | |
|---|---|
| Adenosine phosphate | 0.5 |
| Tranexamic acid | 3 |
| Glycerin | 1 |
| Dipropylene glycol | 4 |
| Polyethylene glycol 20000 | 1 |
| Ethanol | 7 |
| Polyoxyethylene hydrogenated castor oil | 0.4 |
| Polyoxyethylene glyceryl isostearate | 0.2 |
| Pentaerythrityl tetraethylhexanoate | 0.2 |
| Sodium tripolyphosphate | 0.1 |
| Aminohydroxymethylpropanediol | Suitable amount |
| Preservative | Suitable amount |
| Flavor | Suitable amount |
| Purified water | Balance |

### <Manufacturing Method>

According to a conventional method, the water-soluble ingredients were dissolved in purified water to provide an aqueous phase. The water-insoluble ingredients were mixed with the surfactants with heating, and the resulting mixture was gradually added to the aqueous phase to provide the white-turbid lotion.

### Example 43: Beauty Essence

### <Ingredients> % by Weight

| | |
|---|---|
| Adenosine phosphate | 1 |
| Tranexamic acid | 2 |
| Ascorbic acid glucoside | 2 |
| Glycerin | 8 |
| Sorbitol | 2 |
| Oligohyaluronic acid | 0.1 |
| Xanthan gum | 0.2 |
| Quince seed gum | 0.1 |
| Polyoxyethylene hydrogenated castor oil | 0.3 |
| Etidronic acid | 0.03 |
| Aminomethylpropanol | Suitable amount |
| Preservative | Suitable amount |
| Antioxidant | Suitable amount |
| Flavor | Suitable amount |
| Purified water | Balance |

### <Manufacturing Method>

According to a conventional method, the water-soluble ingredients were dissolved in purified water to provide a main phase. The water-insoluble ingredients mixed with the surfactant were added to the main phase to provide the beauty essence.

### Formulation Example 44: Beauty Essence Gel

| | |
|---|---|
| Adenosine phosphate | 1 |
| Tranexamic acid | 1.5 |
| Carboxyvinyl polymer | 0.2 |
| PEG-240/HDI copolymer bis-decyltetradeceth-20 ether | 1 |
| Xanthan gum | 0.2 |
| Polyoxyethylene polyoxypropylene | 0.3 |
| decyl tetradecyl ether | |
| Diethoxyethyl succinate | 1.5 |
| Butylene glycol | 5 |
| Glycerin | 3 |
| Sodium tripolyphosphate | 0.1 |
| Aminohydroxymethylpropanediol | Suitable amount |
| Preservative | Suitable amount |
| Antioxidant | Suitable amount |
| Flavor | Suitable amount |
| Purified water | Balance |

### <Manufacturing Method>

The carbomer was dispersed in purified water, and then the other water-soluble ingredients were added thereto and dissolved by warming to provide a main phase. The water-insoluble ingredients mixed with the surfactant were added to the main phase. The resulting mixture was mixed uniformly and deaerated to provide the desired beauty essence gel.

### Formulation Example 45: Gel Emulsion

### <Ingredients> % by Weight

| | |
|---|---|
| Adenosine Phosphate | 0.5 |
| Tranexamic Acid | 2 |
| Carboxyvinyl polymer | 0.2 |
| Acrylate/alkyl methacrylate copolymer | 0.5 |
| Glycerin | 6 |
| Dipropylene glycol | 4 |
| Cyclopentasiloxane | 5 |
| Dimethicone | 1 |
| Dimethiconol | 0.2 |
| Aminopropyl dimethicone | 0.1 |
| Liquid isoparaffin | 2 |
| Sodium metaphosphate | 0.4 |
| Arginine | Suitable amount |
| Preservative | Suitable amount |
| Antioxidant | Suitable amount |
| Flavor | Suitable amount |
| Purified water | Balance |

### <Manufacturing Method>

The water-soluble polymers and other water-soluble ingredients were dissolved in purified water with warming to provide an aqueous phase. The oil phase, in which the oil-soluble ingredients were dissolved by warming, was dispersed in the aqueous phase and mixed with a disper, and emulsified. After mixing uniformly, the mixture was deaerated to provide the desired gel emulsion.

### Formulation Example 46: Emulsion

### <Ingredient> % by Weight

| | |
|---|---|
| Adenosine phosphate | 0.5 |
| Tranexamic acid | 2 |
| Glycerin | 5 |
| Butylene glycol | 3 |
| Polyoxyethylene hydrogenated castor oil | 3 |
| Pentaerythrityl tetraethylhexanoate | 4 |
| Phytosteryl/behenyl/octyldodecyl lauroyl glutamate | 0.5 |
| Xanthan gum | 0.4 |
| EDTA-2Na | 0.3 |
| Aminomethylpropanediol | Suitable amount |
| Preservative | Suitable amount |
| Antioxidant | Suitable amount |
| Flavor | Suitable amount |
| Purified water | Balance |

### <Manufacturing Method>

The water-soluble polymer and other water-soluble ingredients were dissolved in purified water with warming to provide an aqueous phase. The oil phase, in which the oil-soluble ingredients were dissolved by warming, was dispersed in aqueous phase and mixed with a disper, and emulsified. After mixing uniformly, the mixture was deaerated to provide the desired emulsion.

### Example 47: Cream

### <Ingredients> % by Weight

| | |
|---|---|
| Adenosine phosphate | 1.5 |
| Tranexamic acid | 2 |
| Glycerin | 5 |
| Trehalose | 1 |
| Polyoxyethylene glyceryl monoisostearate | 2 |
| Glyceryl stearate | 3 |
| Sodium stearoyl glutamate | 0.5 |
| Triethylhexanoin | 8 |
| Liquid paraffin | 7 |
| Vaseline | 1 |
| Dimethicone | 0.5 |
| Behenyl alcohol | 3 |
| Stearic acid | 2 |
| Polyvinyl alcohol | 0.5 |
| Hydroxyethyl cellulose | 0.5 |
| Phytic acid | 0.4 |
| Aminohydroxymethylpropanediol | Suitable amount |
| Preservative | Suitable amount |
| Antioxidant | Suitable amount |
| Flavor | Suitable amount |
| Purified water | Balance |

### <Manufacturing Method>

The water-soluble polymers and other water-soluble ingredients were dissolved in purified water with warming to provide an aqueous phase. The oil phase, in which the oil-soluble ingredients were dissolved by warming, was dispersed in the aqueous phase and mixed with a disper, and emulsified. After mixing uniformly, the mixture was deaerated to provide the desired cream.

1. A composition for external use, comprising:
Ingredient (A): an adenosine phosphate and/or a salt thereof; and
Ingredient (B): tranexamic acid and/or a salt thereof,
and further comprising
Ingredient (C): a chelating agent selected from edetic acid or a salt thereof, a phosphate compound, and a mixture thereof; and/or
Ingredient (D): a pH adjuster which is an organic alkali compound.

2. The composition according to claim 1, wherein the adenosine phosphate and/or a salt thereof is an adenosine monophosphate and/or a salt thereof.

3. The composition according to claim 1, wherein the phosphate compound is selected from tripolyphosphoric acid or a salt thereof, metaphosphoric acid or a salt thereof, etidronic acid or a salt thereof, phytic acid or a salt thereof, and a mixture thereof.

4. The composition according to claim 1, wherein Ingredient (C) is the phosphate compound selected from tripolyphosphoric acid or a salt thereof, metaphosphoric acid or a salt thereof, etidronic acid or a salt thereof, and a mixture thereof.

5. The composition according to claim 1, wherein Ingredient (D) is selected from aminohydroxymethylpropanediol, aminomethylpropanol, aminomethylpropanediol, arginine, triethanolamine, and a mixture thereof.

6. The composition according to claim 1, wherein Ingredient (C) is comprised in a weight ratio of (Ingredient (A)+Ingredient (B)):Ingredient (C)=1:0.0001 to 0.5.

7. The composition according to claim 1, which comprises Ingredient (D) and has pH of 5.5 to 7.5.

8. The composition according to claim 1, wherein Ingredient (D) is comprised in a weight ratio of Ingredient (A):Ingredient (D)=1:0.00001 to 4.

9. The composition according to claim 1, which comprises 0.01 to 10% by weight of Ingredient (A).

10. The composition according to claim 1, which comprises 0.01 to 10% by weight of Ingredient (B).

11. The composition according to claim 1, further comprising Ingredient (E): bis-ethoxydiglycol cyclohexane dicarboxylate, diethoxyethyl succinate, highly polymerized polyethylene glycol, highly polymerized silicone, pullulan, polyvinylpyrrolidone, an organic fine particle, biosaccharide gum-1, or a mixture thereof.

12. A method for producing a composition for external use, comprising mixing Ingredient (A): an adenosine phosphate and/or a salt thereof and Ingredient (B): tranexamic acid and/or a salt thereof, in the presence of Ingredient (C): a chelating agent selected from edetic acid or a salt thereof, a phosphate compound, and a mixture thereof; and/or Ingredient (D): a pH adjuster which is an organic alkali compound.

13. A method for suppressing discoloration of a composition for external use which comprises Ingredient (A): an adenosine phosphate and/or a salt thereof and Ingredient (B): tranexamic acid and/or a salt thereof,
comprising using Ingredient (C): a chelating agent selected from edetic acid or a salt thereof, a phosphate compound, and a mixture thereof; and/or Ingredient (D): a pH adjuster which is an organic alkali compound.
